(19) United States
(12) Patent Application Publication
(10) Pub. No.: US 2021/0386722 A1
(43) Pub. Date: Dec. 16, 2021

(54) Treatment of Covid-19 with Nitroxides

(71) Applicant: Peter Herbert Proctor, Houston, TX (US)

(72) Inventor: Peter Herbert Proctor, Houston, TX (US)

(21) Appl. No.: 17/354,068

(22) Filed: Jul. 9, 2021

(51) Int. Cl. A61K 31/445; A61P 31/14

(52) U.S. Cl. CPC A61K 31/445; A61P 31/14

(57) ABSTRACT

Nitrone, nitroso, and nitroxide drugs and their corresponding hydroxylamine reduction products are claimed for the acute and chronic treatment of Covid-19 and its variants.

Priority is claimed to provisional patent application No. 63/046,725, dated Jul. 1, 2020

### BACKGROUND

### Prior Art

This patent relates to the treatment of a respiratory coronavirus SARS-COV-2 infection, (“Covid-19”) with a nitroxide drug. This previously-unknown disease has recently caused a massive world-wide epidemic with millions dead and a partial shutdown of the world economy (Dong, et al, 2020). Post-acute Covid-19 is also a significant cause of morbidity and mortality. While vaccines have proved efficacious in preventing Covid-19 infections, to date, systemic treatments such as the viral replicase-inhibitor Remdisivir or the antimalarial drug hydroxychloroquine have proven ineffective or only marginally-effective, at best (Harrington, et al, 2021—Note: post-priority date and for information purposes only). Thus, there is an urgent need for an orally-effective, non-toxic, and relatively inexpensive agent to treat Covid-19. Similarly, “Oral drugs that could be taken at home early in the course of disease would be powerful tools for battling the pandemic and saving lives,” Dr. Anthony Fauci. Likewise, “We urgently need additional effective, accessible treatments for COVID-19. An oral drug that prevents SARS-CoV-2 from replicating would be an important tool for reducing the severity of the disease.” Dr. Diana W. Bianchi, director of the NIH's National Institute of Child Health and Human Development.

In general, the coronaviruses are a broad and numerous class of RNA viruses causing a variety of diseases in humans and animals (Cui et al, 2019). With the recent notable exception of Covid-19, and the related SARS-COV-1 and MERS viruses, human respiratory coronavirus infections are generally mild and self-limited and, as a cause of “colds”, are second only to the adenoviruses, accounting for an estimated 10-30% of colds. Consequently, per the CDC's “Common Covid Factsheet” (2020) “. . . Most people get infected with one or more of these viruses at some point in their lives.”

Similarly, oxidative processes, modulated both by direct chemical attack and by modulation of biological processes by redox processes or “Redox Signaling” have long been known to play a key role in the etiology of inflammatory diseases, cancer, and degenerative diseases (Proctor, 1989). This includes lung diseases such as pulmonary fibrosis and ARDS (Proctor, 1989). Examples of such redox-effector molecules include superoxide free radical, hydrogen peroxide, their daughter products, and the like. For example, superoxide dismutase enzymes (“SOD's”) catalyze the dismutation of superoxide free radicals to peroxide and water. Such SOD's, as well as small-molecule SOD-mimetic nitroxide spin labels such as Tempol, are reported to have antiinflammatory activity in a variety of human and animal diseases, as well as other more specific actions likely connected to the action of superoxide and its daughter products as cellular messengers, as well as possible direct effects of the molecules themselves. One example is modulation of the hair cycle, another is cancer cell growth and metastasis, where nitroxides may paradoxically act as pro-oxidants, as well as antioxidants. For reviews, see Huber W, (1981), Proctor P (1989), Wilcox C S.(2010), Zarling J A, et al (2015), and Lewandowski M and Gwozdzinski K. (2017), which are incorporated by reference.

Thus, in the mid 1980's we were the first to discover the pharmaceutical actions of nitroxide spin labels and subsequently patented several such compounds for medicinal applications. E.g., the original drug utility for Tempol (4-hydroxytempo) is for the treatment of alopecia, with a priority date of Jul. 7, 1985 (U.S. Pat. No. 5,728,714A).

Similarly, after unexpectedly finding it to ameliorate virus-induced upper respiratory symptomology, we have long used Tempol to treat colds and influenza. Thus, Proctor patent application US20120115905A1, corresponding to U.S. Pat. No. 8,778,969B2, teaches the use of Tempol to treat influenza. Unfortunately, due to limited resources and the dismal prospect of commercialization in the face of the enormous cost of regulatory approval, these antiviral claims were not followed up. Similarly, while we have continued to investigate in this area, colds, a significant cause of which is a coronavirus infection (Paules, et al, 2020), were omitted from the '905 application as being of little commercial interest. Nor, have we ever noted this possible application in the literature. Thus, the final '969 patent only claims the use of Tempol and allied compounds for the treatment of fibrocystic disease of breast. Unfortunately, this also has not been commercialized. Similarly, although the condition of “Radiologically-Dense Breasts” is the single most important clinical precurser for breast cancer, our patent U.S. Pat. No. 10,028,943 (claiming treatment of this condition with Tempol and its sister nitroxide spin labels), has also not been picked up for further development. Similar discoveries from (e.g.) the National Cancer Institute claiming the use of Tempol or its derivatives to treat, e.g., various cancers have also not been commercialized (for reviews, see, Wilcox C S. (2010), and Zarling J A, et al (2015) , and Lewandowski M and Gwozdzinski K. (2017)). Likewise, the one attempt by a large drug company to commercialize the nitrone antioxidant drug NXY-059 for the treatment of stroke ended up a last-minute failure (Proctor and Tamborello, 2007). This wiped several billion dollars off the net asset value of the sponsoring drug company, Astrazenica.

More broadly, because of this latter experience and the general failure of promising results in animal studies to replicate in humans, for a time major drug companies were simply uninterested in any antioxidant drug. Stated-simply, positive results with antioxidant drugs in animal studies have not translated to humans. Thus, Traystman, R J (2010) teaches with respect to antioxidant neuroprotective drugs, “There are hundreds, perhaps thousands of neuroprotective drugs that have been used in animal models. So, if you were a mouse or a rat, and experienced a stroke or cardiac arrest, we would know just what to do for you. But, essentially none of these pharmacological agents have demonstrated usefulness in humans even though they have been shown to be successful in preclinical animal trials” . Similarly, U.S. Pat. No. 10,441,568B2, and Application #20180085347A1 claim the use of certain cyclic nitroxides (but not Tempol or its derivatives) for the treatment of disorders of the respiratory tract of experimental animals. This also includes bleomycin-induced pulmonary fibrosis, likely due to reactive oxygen species or “ROS” (Proctor, 1989, p215). In this regard, we have previously shown that the systemic toxicity of bleomycin can be ameliorated by co-administration of catalase and Orgotein, the pharmaceutical form of superoxide dismutase (McGinness, et al,1982). Similarly, in a speculative list of possible applications, our '714 patent includes treatment of pulmonary fibrosis as a potential utility for Tempo. However, nowhere do patents '724 or '586 or the '347 application teach treatment of coronavirus infection. On the other hand, Tsuhako et al (2010) report that Tempol ameliorates murine viral encephalomyelitis induced by a nonrespiratory coronavirus unrelated to Covid-19.

Significantly, well after our present priority date, Maio et al (2021) reported that, by oxidizing iron-sulfur centers in the SARS-COV-2 replicase (RNA-Dependent RNA polymerase), Tempol inactivates this key enzyme and markedly inhibits viral replication in vitro. Further, it does this at potentially “physiological” Tempol concentrations, implicating that this agent may be directly antiviral. On the other hand, such direct antiviral action is contrary to the standard model of Tempol as an in vivo antioxidant and SOD-mimetic. In addition to bearing on bearing on “obviousness”, such unexpected antiviral activity may explain our findings of a negative PCR test for SARS-COV-2 RNA at day 11 of the illness and why anti-SARS-COV-2 IGG antibodies were negative.

Note: Maio et al was published well after the applicable priority date for this patent application and is provided here for information purposes alone. Nor are our claims dependent upon any specific mechanism of action or theory for validity. Similarly, diseases are very complicated. Thus, efficacy in one specific symptom or sign in one distinct disease does not confer “obviousness” that a drug will work in another or even that results in preclinical studies will translate to humans. This is especially the case where a particular disease was completely unknown until recently and the drug is an antioxidant drug.

### BRIEF SUMMARY OF THE INVENTION

The present invention is directed to methods that treat, inhibit, or slow the development of the symptoms of coronavirus SARS-CoV-2 infection (“Covid-19”), both acutely and longer term. The inventive methods comprise the administration both acutely and chronically of pharmaceutical preparations comprising nitrone, nitroxide and nitroso compounds and their corresponding reduction products orally, intraorally, intranasally, topically, by nasal insufflation, inhalation, systemically by injection, or by local rectal administration.

### DRAWINGS

Not applicable

### DESCRIPTION OF THE INVENTION

### Definitions

The terms “nitroxide” , “nitrone”, and “nitroso” are used herein to describe molecules comprising an oxygen and a nitrogen atom directly bound to each other. These compounds may be a electron donors or acceptors. Depending on their oxidation state, these compounds may comprise stable nitroxyl free radicals including precursors (such as the N—H form), and derivatives thereof including their corresponding hydroxylamine derivative (N—OH), where the oxygen atoms are replaced with a hydroxyl group and/or exist in a hydrogen halide form. Nitroxides and nitrones of the invention may be administered to a system, such as a human, and act to modulate oxidation and reduction reactions by donating or accepting an electron. Other mechanisms may include formation of charge-transfer complexes as well as by “redox signaling” or modulation of redox-signaling-mediated processes.

Stability of unpaired electrons on such compounds is typically-provided at the nitrogen nucleus by two adjacent carbon atoms that may be substituted with strong electron donor groups. With the partial negative charge on the oxygen of the N—O bond, the two adjacent carbon atoms together localize the unpaired electron on the nitrogen nucleus.

Nitroxides and nitrones generally may have either a heterocyclic or a linear structure. In an in vivo environment a nitroxide may react with a first superoxide to form oxoammonium (as an electron donor) and then react with a second superoxide to re-form the nitroxide (as an electron acceptor). For review, see Wilcox C S. (2010)

The terms “treat,” “treatment” and the like are used herein to generally mean obtaining a desired pharmacological and/or physiological effect in humans or other animals. A treatment is an approach for obtaining beneficial or desired clinical results. While the claims are not dependent on any specific mechanism, in the present case, these clinical results include but are not limited to decreasing undesirable effects of reactive oxygen species (ROS) and oxidative stress in general, as well as modulating more specific messenger processes such as “redox signaling”. The effect may be therapeutic in terms of a partial or complete cure of the disease and/or adverse effect attributed to the disease. In general, methods of the invention may be applied to a variety of different areas including the skin, mucus membranes including those in the GI tract, nose, throat, mouth, vaginal cavity, ocular surfaces, as well as the surfaces of the lungs and the surfaces of the vascular system as well as systemically by means of intravenous, intraocular, intramuscular, transdermal, sublingual, by insufflation, and/or intraoral administration. “Treatment” as used herein covers any treatment of such a symptom or disease in a mammal, particularly a human, and includes:

(I.a) inhibiting the disease, i.e. arresting it's development; or

(I.a) relieving the disease and/or it's symptom, i.e. causing regression of the disease and/or the symptoms caused by the disease.

Exemplary compounds include, but are not limited to, DEPMPO (5-(Diethoxyphosphoryl)-5-methyl-1 -pyrroline N-oxide), TEMPO (2,2,6,6-tetramethyl-1-piperidinyl-1-oxyl), 4-Amino-TEMPO, 4-hydroxy-TEMPO (TEMPOL), DMPO (5,5-dimethylpyrroline-N-oxide), EMPO (2-Ethoxycarbonyl-2-methyl-3,4-dihydro-2H-pyrro-1-oxide), POBN (alpha-(4-pyridyl-1-oxide)-N-tert-butylnitrone), TEMPONE (4-Oxo-2,2,6,6-tetramethylpiperidine-1-oxyl 4-Oxo-TEMPO), TMIO ,3,3,5,5 tetramethyl-1-pyrolline-N-oxide (TMPO), M3PO (2,5,5-trimethyl-1-pyrroline-N -oxide), M4PO (3,3,5,5-tetramethyl-1-pyrroline-N-oxide), TMPO (3,3,5,5 tetramethyl-1-pyrolline-N-oxide), PBN (1-alpha-phenyl-tert-butyl nitrone), and MNP (2-methyl-2-nitrosopropane), and MitoTempol (2,2,6,6-Tetramethyl-4-[[5-(triphenylphosphonio)pentyl]oxy]-1-piperidinyloxy bromide), as well as their corresponding hydroxylamine derivatives and prodrugs (e.g., Zarling et al, 2015). The various sulfone (e.g., NXY-059, disulfonyl PBN), hydroxyl, and other derivatives such as esters, peptides, hydroxyl, hydroxylamines, nitrones, carboxyls, and so forth are also claimed.

Preferred examples of the type of hydroxylamine compounds suitable for use in the present invention are TEMPOL-H (the hydroxylamine reduced form of the nitroxide 4-hydroxy-2,2,6,6-tetramethylpiperidin-1-yloxy), TEMPO-H (the hydroxylamine reduced form of the nitroxide 2,2,6,6- tetramethylpiperidin-1-yloxy) and OXANO-H (2-ethyl-2,4,4-trimethyloxazolidine, which is the reduced form of oxano, 2-ethyl-2,4,4-trimethyloxazolidin-3-yloxy), as well as MitoTempol-OH. Other hydroxylamine compounds suitable for use in the present invention include, but are not limited to, those disclosed by Hahn et al. (1998, supra; 2000, supra), Samuni et al. (2001, supra); and in U.S. Pat. No. 5,981,548 to Paolini, et al. (disclosing certain N-hydroxylpiperidine esters and their use as antioxidants in a number of contexts);

U.S. Pat. No. 4,404,302 to Gupta et al. (disclosing the use of certain N-hydroxylamines as light stabilizers in plastics formulations); and U.S. Pat. No. 5,462,946 to Mitchell et al. (disclosing certain nitroxides deriving from substituted oxazolidines for protection of organisms from oxidative stress). Most of the above-referenced compounds have not been known heretofore for administration to humans. Certainly, none of them has been known for use in the treatment of respiratory coronavirus infection, much less Covid-19. Suitable reducing agents include, but are not limited to: ascorbic acid, lipoic acid, cystine, purines and derivatives such as acetylcysteine, uric acid and other oxyxanthines, methionine, homocysteine, NADPH, and NADH.

### EXAMPLES

### Preparation of a Therapeutic Solution of TEMPOL-H

The reduced form of TEMPOL (TEMPOL-H) or equivalent pharmacologically-active spin label/spintrap is prepared by mixing together 6 grams of TEMPOL or a pharmacologically effective amount of another spin-label or spin trap such as methynitrosopropane, 20 grams of ascorbic acid or other reducing compound in 100 ml of distilled water. The formulation is used as is. The solution is slightly-bitter. The formulation can be administered diluted in a suitable liquid such as juice, tea, or coffee. An equivalent dry form as 20 mg of tempol mixed with 100 mg of ascorbic acid or equivalent reducing substance can be easily prepared in capsule or tablet form.

### Treatment of Coronavirus

A septuagenarian male experienced sudden frightening onset of severe headache, marked nausea and vomiting, pharyngitis, severe upper respiratory congestion, fever, chills, myalgia, weakness, malaise, and mild to moderate pulmonary congestion. O2 saturation=96%. Patient had been taking Tempol off-and-on for years for cancer prophylaxis and for its possible antiaging activity, as well as some miscellaneous actions such as treatment of periodontitis and osteoarthritis. But, concerned that it would interfere with their activity, he had stopped Tempol about 6 weeks before receiving influenza (Fluvax) and Zoster (Zostavax) immunizations. The presumptive-diagnosis was “Covid-19”, possibly from a medically-related exposure.

Treatment: Nausea and vomiting continued without respite for several hours. Anti-nausea tablets were twice regurgitated intact. Accordingly, treatment was initiated with 30 mg of Tempol PO as the hydroxylamine in a 6% water solution containing 90 mg vitamin-C. Patient kept this down. Along with Tempol-zinc gargles for pharyngitis, intranasal treatment was then initiated with a medication-soaked cotton swab and insufflation, repeated approximately q 6 h as symptoms returned. This was followed by similar doses of Tempol-H three and six hours later, repeated 2-3 times a day, PO for the next ten days. Subsequent intranasal treatment was also repeated 1-3 times a day for 10 days, as needed, titrating to relief of oropharyngeal symptoms as they recurred. On days 2-3, treatment was briefly initiated with hydroxychloroquine, zinc, doxycycline, ivermectin, and famotidine, then withdrawn.

Results: On initiation of treatment, vomiting ceased almost immediately and did not recur. Within one to two hours, headache eased significantly, and the patient felt objectively better. Nasal congestion also quickly reversibly-eased upon repeated intranasal treatment. Gargles provided relief from pharyngitis. Pulmonary congestion also cleared somewhat. However, the patient experienced “vivid dreaming” that night and several subsequent ones, as well as overall “spaciness”.

By days 5-7 remaining symptoms were mild reversible-on-treatment nasal congestion, pharyngitis, and malaise. Excepting slight malaise, patient was essentially asymptomatic by days 8-9. PCR for SARS-COV-2 RNA was negative at day 10. While not surprising considering the clinical course, in retrospect, this may have signified some direct antiviral effect of the application of Tempol directly to the upper respiratory mucosa and oropharynx by insufflation, inhalation, or gargling. Interestingly, although the diagnosis is reasonable secure, IGG antibodies against the virus were also negative at days 11 and 58, also possibly reflecting some antiviral activity. While heavily exposed to patient 1, A 60-year-old woman treated similarly with Tempol 60 mg/day for fibrocystic disease of breast did not develop clinically-significant coronavirus disease. After patient discontinued treatment for several weeks, subsequent putative recurrent post-acute symptoms of Covid-19 such as malaise, myalgia, and shortness of breath appeared. These also responded reversibly to further Tempol treatment, as above, with the addition of direct lung inhalation of 0.1 ml of the 6% Tempol or Tempol-H solution, all titrated to a clinical response.

Alternate forms of oral administration such as tablets, pills, or capsules are also effective. As noted, a gargle composed of 0.5% Tempol and 0.05% zinc sulfate in water provides relief of pharyngeal discomfort. Parental modes of administration such as intravenous, intramuscular, subcutaneous, intraperitoneal, transrectal, or by direct inhalation into lungs are also possible, as are topical modes of administration such as in lotions, creams, gels, and topically-compatible suspensions and solutions. Tempol itself is effective at the same doses, but may have increased side-effects at higher doses, e.g., due to oxidation of reducing agents or production of hydrogen peroxide.

Such methods that are routine in the art, and may vary with the needs of individual subjects.

The present invention is not limited to the embodiments described and exemplified above, but is capable of variation and modification within the scope of the appended claims. Also, the claims are not bound by any suggested possible mechanism of action and are independent thereof.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention. Likewise, each claim and indication stands independent of the patentability or patent status of any other claim and indication.

What is claimed is:

1. Compound for the treatment of Covid-19, wherein the compound is TEMPO (2,2,6,6-tetramethyl-1-piperidinyl-1-oxyl), 4-Amino-TEMPO, TEMPOL (4-hydroxy-TEMPO), TEMPONE (4-Oxo-2,2,6,6-tetramethylpiperidine-1-oxyl 4-Oxo-TEMPO), 2-Methyl-2-nitrosopropane, MitoTEMPOL (2,2,6,6-Tetramethyl-4-[[5-(triphenylphosphonio)pentyl]oxy]-1-piperidinyloxy bromide), or as well as their corresponding hydroxylamine derivatives, as well as their corresponding prodrugs, to include OT-551 (1-hydroxy-4-cyclopropanecarbonyloxy-2,2,6,6-tetramethylpiperidine hydrochloride), or OT-440 (4-(4-(1-hydroxy-2,2,6,6-tetramethylpiperidin-4-yloxy)-1,2,5-thiadiazol-3-yl) morpholine hydrochloride.

2. Compound for the treatment of Covid-19, wherein the compound is TEMPO (2,2,6,6-tetramethyl-1-piperidinyl-1-oxyl), 4-Amino-TEMPO, TEMPOL (4-hydroxy-TEMPO), TEMPONE (4-Oxo-2,2,6,6-tetramethylpiperidine-1-oxyl 4-Oxo-TEMPO), 2-Methyl-2-nitrosopropane, MitoTEMPOL (2,2,6,6-Tetramethyl-4-[[5-(triphenylphosphonio)pentyl]oxy]-1-piperidinyloxy bromide), as well as their corresponding prodrugs, to include OT-551 (1-hydroxy-4-cyclopropanecarbonyloxy-2,2,6,6-tetramethylpiperidine hydrochloride), or OT-440 (4-(4-(1-hydroxy-2,2,6,6-tetramethylpiperidin-4-yloxy) -1,2,5-thiadiazol-3-yl)morpholine hydrochloride, wherein said compound is liable to be acutely and/or chronically administered at a dose comprised 0.01 mg/kg to 300 mg/kg, in particular from 0.1 mg/kg to 20 mg/kg.

3. Compound for the treatment of Covid-19, wherein the compound is TEMPO (2,2,6,6-tetramethyl-1-piperidinyl-1-oxyl), 4-Amino-TEMPO, TEMPOL (4-hydroxy-TEMPO), TEMPONE (4-Oxo-2,2,6,6-tetramethylpiperidine-1-oxyl 4-Oxo-TEMPO), 2-Methyl-2-nitrosopropane, or MitoTEMPOL (2,2,6,6-Tetramethyl-4-[[5-(triphenylphosphonio)pentyl]oxy]-1-piperidinyloxy bromide), as well as their corresponding hydroxylamine derivatives, as well as their corresponding prodrugs to include OT-551 (1-hydroxy-4-cyclopropanecarbonyloxy-2,2,6,6-tetramethylpiperidine hydrochloride), or OT-440 (4-(4-(1-hydroxy-2,2,6,6-tetramethylpiperidin-4-yloxy)-1,2,5-thiadiazol-3-yl) morpholine hydrochloride, wherein the mode of administration is orally, intravenously, intramuscularly, transcutaneously, as a prodrug, subcutaneously, transrectally, intraocularly, by nasal insufflation, as a gargle to the oropharynx, or by inhalation into the lungs, using clinically-appropriate formulations such as in parenteral or topical solutions, dry powders, suspensions, gels, ointments, powders, or creams.

4. Compound for the treatment of Covid-19, wherein the compound is TEMPO (2,2,6,6-tetramethyl-1-piperidinyl-1-oxyl), 4-Amino-TEMPO, TEMPOL (4-hydroxy-TEMPO), TEMPONE (4-Oxo-2,2,6,6-tetramethylpiperidine-1-oxyl 4-Oxo-TEMPO), 2-Methyl-2-nitrosopropane, MitoTEMPOL (2,2, 6,6-Tetramethyl-4-[[5-(triphenylphosphonio)pentyl]oxy]-1-piperidinyloxy bromide), as well as their corresponding hydroxylamine derivatives, as well as their corresponding prodrugs, to include OT-551 (1-hydroxy-4-cyclopropanecarbonyloxy-2,2,6,6-tetramethylpiperidine hydrochloride), or OT-440 (4-(4-(1-hydroxy-2,2,6,6-tetramethylpiperidin-4-yloxy)-1,2,5-thiadiazol-3-yl) morpholine hydrochloride, administered along with a reducing agent.
